(19) United States
(12) Patent Application Publication
(10) Pub. No.: US 2002/0132339 A1
(43) Pub. Date: Sep. 19, 2002

(54) Apparatus for capturing suspended microorganisms

(76) Inventor: Noriyasu Takashima, Yokohama (JP)

Correspondence Address: Noriyasu Takashima, 4-19-7, Higashiterao, Tsurumi, Yokohama, Kanagawa, JP

(21) Appl. No.: 09/805,693

(22) Filed: Mar. 13, 2001

(51) Int. Cl. C12M 1/26

(52) U.S. Cl. 435/309.1; 435/287.1; 73/28.05

(57) ABSTRACT

The present invention provides a capturing apparatus that prevents contamination caused by a porous body coming in contact with a desk and so forth when transferring a Petri dish or other container.

The present invention discloses an apparatus for capturing suspended microorganisms comprising: a cap provided so as to be able to be opened and closed freely in a chamber without being separated from said chamber that serves as both an inlet port for outside air and a transfer port for a Petri dish or other container; a porous plate provided in said inlet port of said cap; a holder for said container provided at a position that allows receiving of outside air aspirated from said porous plate; and, a deaerating pump for generating negative pressure in said chamber.

In addition, with respect to the above constitution, the cap is attached to the chamber so that the cap is able to be opened and closed freely while maintaining a roughly horizontal orientation at all times relative to the chamber without being separated from the chamber.

### BACKGROUND OF THE INVENTION AND RELATED ART STATEMENT

This invention relates to an apparatus for capturing microorganisms suspended in the air, and is characterized by being composed so as to prevent contamination by microorganisms and so forth on a desktop by separating a porous body from a capturing apparatus during transfer of microorganisms between Petri dishes and other containers.

In a so-called Andersen type of suspended microorganism capturing apparatus, a porous body for jetting outside air aspirated from an inlet port towards an agar medium is arranged above a Petri dish. The porous body is provided in a cap for removing and inserting the Petri dish. Thus, although the cap must be removed when removing and inserting the Petri dish, the cap may be inadvertently or carelessly placed on a desk and so forth.

However, since the cap is provided with a large number of holes that serve as pathways for outside air containing suspended microorganisms to be captured, this leads to the undesirable result of contamination of the desk and so forth due to contact with the cap. One of the inherent objectives of capturing microorganisms is mainly to accurately determine the number of suspended microorganisms in the air in an office or other general environment and in a clean room.

In consideration of the problems described above, the object of this invention is to provide a capturing apparatus that eliminates contamination caused by a porous body coming in contact with a desk and so forth during transfer of a Petri dish or other container.

### SUMMARY OF THE INVENTION

The above object is achieved by providing an apparatus for capturing suspended microorganisms comprising: a cap provided so as to be able to be opened and closed freely in a chamber without being separated from said chamber that serves as both an inlet port for outside air and a transfer port for a Petri dish or other container; a porous plate provided in said inlet port of said cap; a holder for said container provided at a position that allows receiving of outside air aspirated from said porous plate; and, a deaerating pump for generating negative pressure in said chamber.

Although the cap is able to be opened and closed freely, since it is provided so as not to be separated from the chamber, even if the cap is opened and closed immediately after transferring the Petri dish or other container, it is not placed on a desk and so forth. Thus, there is little risk of contamination by the cap as well as the porous plate by this route.

In addition, the above of object of the present invention is also achieved by providing an apparatus for capturing suspended microorganisms comprising: a cap that is maintained roughly in the horizontal position at all times in a chamber, is provided so as to be opened and closed freely without being separated from said chamber and serves as both an inlet port for outside air and a transfer port for a Petri dish or other container; a porous body provided in said inlet port of said cap; a holder for said container provided at a position that is able to receive outside air aspirated from said porous body; and, a deaerating pump for generating negative pressure in said chamber.

By providing a porous plate fixed in the inlet port of the cap, the porous plate does not come out of the inlet port regardless of the orientation of the cap. However, there also cases in which it is desired to change the arrangement pattern of holes in the porous plate. In such cases, the porous plate should not be fixed in the inlet port. Therefore, a cap is provided in the chamber such that it is maintained roughly in the horizontal position at all times while allowing to be opened and closed freely. Thus, the porous plate does not come out of the inlet port provided that the apparatus is used normally. Furthermore, the arrangement pattern of holes in the porous plate will be described later.

### BRIEF DESCRIPTION OF THE DRAWINGS

The invention disclosed herein will be understood better with reference to the following drawings of which:

FIG. 1 is a schematic drawing of a capturing apparatus of a first embodiment.

FIG. 2 is a cross-sectional view of the vicinity of cap 2.

FIG. 3 is an overhead view of porous plate 20.

FIG. 4 is a schematic drawing depicting the state in which cap 2 has been opened.

FIGS. 5 and 6 are explanatory drawings of the operation during opening and closing of cap 2 in a second embodiment.

FIG. 7 is an overhead view of porous plate 29.

FIG. 8 is a schematic drawing of a capturing apparatus of a third embodiment.

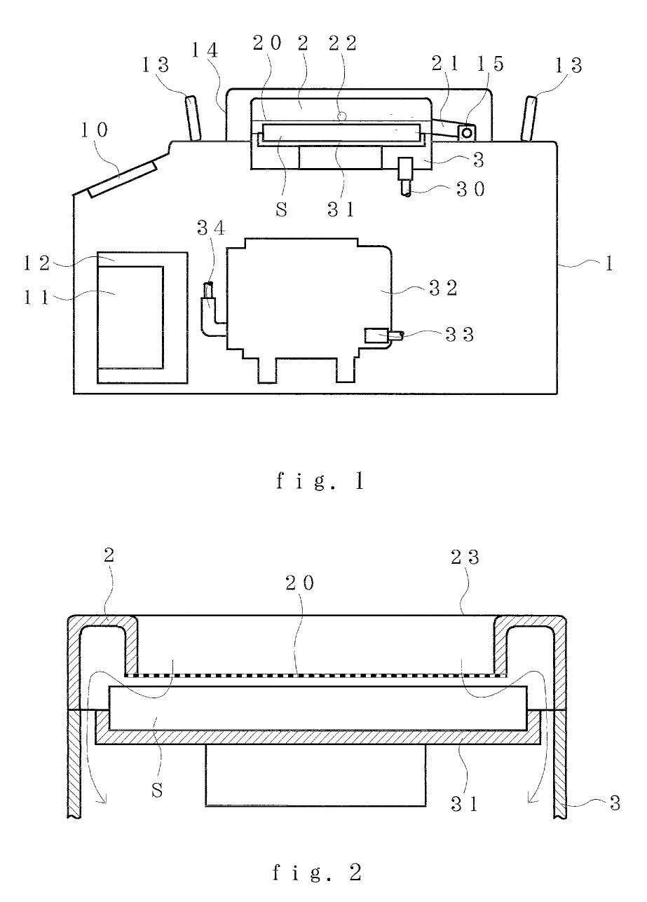

### DETAILED DESCRIPTION OF THE PREFERRED EMBODIMENTS

Although the following provides an explanation of several embodiments of the invention with reference to the drawings, the invention is not limited to these embodiments.

Holder 31 for holding Petri dish S is provided inside chamber 3. Inlet port 34 of vacuum pump 32 for generating negative pressure inside chamber 3 and chamber 3 are connected with pipe 30, and exhaust port 33 of vacuum pump 32 is led to the outside of casing 1 of the capturing apparatus via a different pipe. Reference symbol 11 is a driver circuit, while reference symbol 12 is a power supply circuit. In addition, reference symbol 10 is a membrane switch for emitting an ON/OFF command or other control command for vacuum pump 32.

Cap 2 is attached to the above-mentioned chamber 3 while allowing to be opened and closed freely on the upper surface of casing 1 by arm 21. Arm 21 is attached to cap 2 by shaft 22 and to casing 1 by shaft 15. In addition, when not using capturing apparatus 1, cover 14 made of clear synthetic resin can be placed over cap 2 so as to cover it.

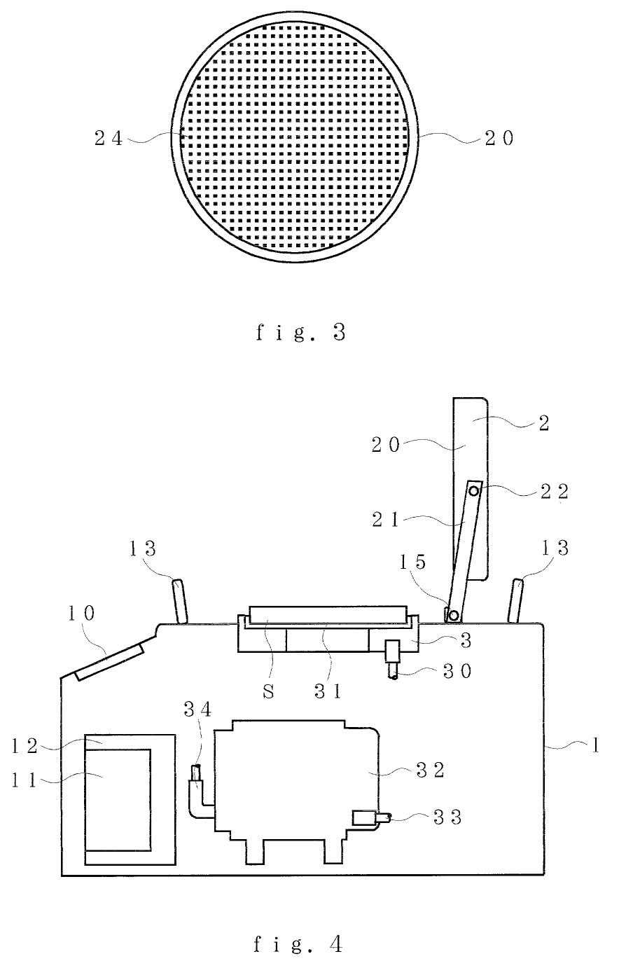

Inlet port 23 is opened in the upper surface of the above-mentioned cap 2, and porous plate 20 is fixed in this inlet port 23. As shown in FIG. 3, this porous plate 20 has pinholes 24 arranged in the form of a grid over the entire surface of a thin iron plate. In addition, porous plate 20 is able to be in close proximity to Petri dish S placed on the above-mentioned holder 31. Thus, when vacuum pump 32 is activated, negative pressure is generated inside chamber 3 and outside air containing microorganisms is forcibly aspirated through the above-mentioned inlet port 23, after which that outside air is blown downward through pinholes 24 in porous plate 20 located at the bottom of inlet port 23. In this manner, outside air collides strongly with the agar medium of Petri dish S causing suspended microorganisms to be adhered to or embedded in the agar medium, after which it passes through the area around holder 31 and is aspirated into pipe 30. In FIG. 2, the movement of air is indicated with arrows.

FIG. 4 shows the state in which cap 2 is opened during transfer of Petri dish S. Although cap 2 is oriented vertically, since porous plate 20 is fixed to cap 2, it does not fall out of cap 2. Thus, there is no risk of contamination of a desktop by miscellaneous microorganisms and so forth since porous plate 20 is not placed on a desktop, and there is no failure in miscellaneous microorganisms entering the medium mixed in with outside air that has passed through cap 24 at high speed. Furthermore, cap 2 can also be placed in the horizontal position depending on the action of shaft 22.

Since the large number of pinholes 24 of porous plate 20 are arranged in the form of a grid, when the agar medium is cultured following entrance of suspended microorganisms, colonies are formed while arranged roughly in the form of a grid. Thus, the number of colonies can be counted more easily than in the case of randomly dispersed colonies, and can also be counted mechanically by pattern recognition using a computer without having to rely on manual counting.

Next, the following provides an explanation of a different embodiment with respect to the attachment of cap 2 to the above-mentioned chamber 3. Although cap 2 is fit in cap holder 35 of chamber 3, bearing 17, provided on the upper surface of capturing apparatus 3, and cap 2 are coupled with two parallel arms 21 and 25, and these arms 21 and 25 are attached to bearing 17 with rotating shafts 15 and 16, respectively, and to both sides of cap 2 with rotating shafts 22 and 26, respectively. Cap 2 is therefore able to be opened and closed freely while maintaining a roughly horizontal orientation relative to cap holder 35 of chamber 3.

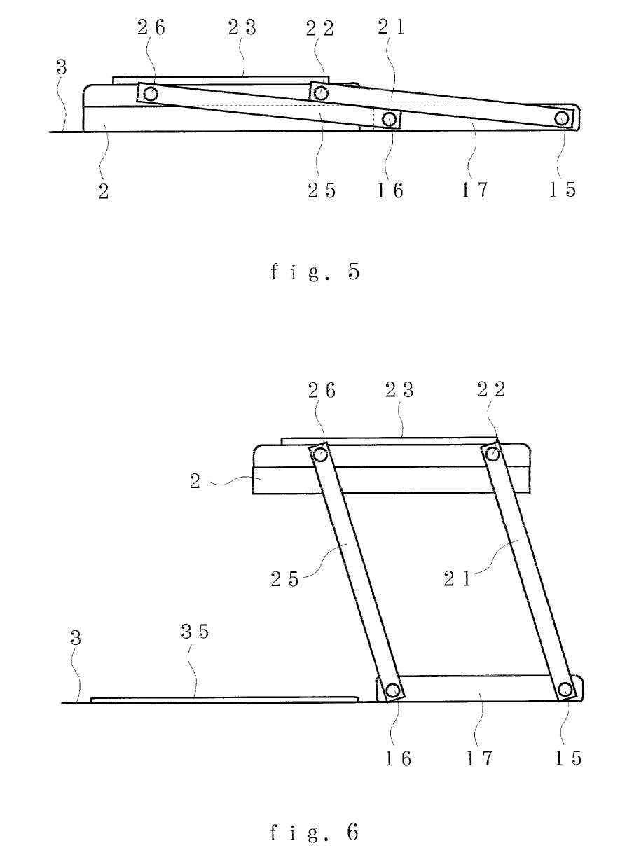

This operating state is expressed in FIGS. 5 and 6. In FIG. 5, the above-mentioned cap 2 is shown in the state of being fit in cap holder 35 of chamber 3. However, when cap 2 is raised, it rises diagonally while maintaining a parallel orientation as shown in FIG. 6. Namely, cap 2 is maintained in the horizontal position at all times.

There are also cases in which it is desired to interchange a plurality of porous plates having different diameters of pinholes or different arrangement patterns for a large number of pinholes. This embodiment is an example of that designed for this type of utilization. For example, in porous plate 29 shown in FIG. 7, a large number of pinholes 200 are arranged in a radial manner from the center of porous plate 29. Since cap 2 is opened and closed while maintaining a roughly horizontal orientation at all times even if this porous plate 29 is freely attached to and removed from cap 2, there are no problems resulting from porous plate 29 falling out of cap 2 and dropping onto the floor resulting in contamination of the floor by miscellaneous microorganisms. Thus, there is no failure in miscellaneous microorganisms entering the medium mixed in with outside air that has passed through pinholes 200 at high speed. Furthermore, a different design in which cap 2 is constantly maintained in the horizontal position in this manner is explained in the following third embodiment.

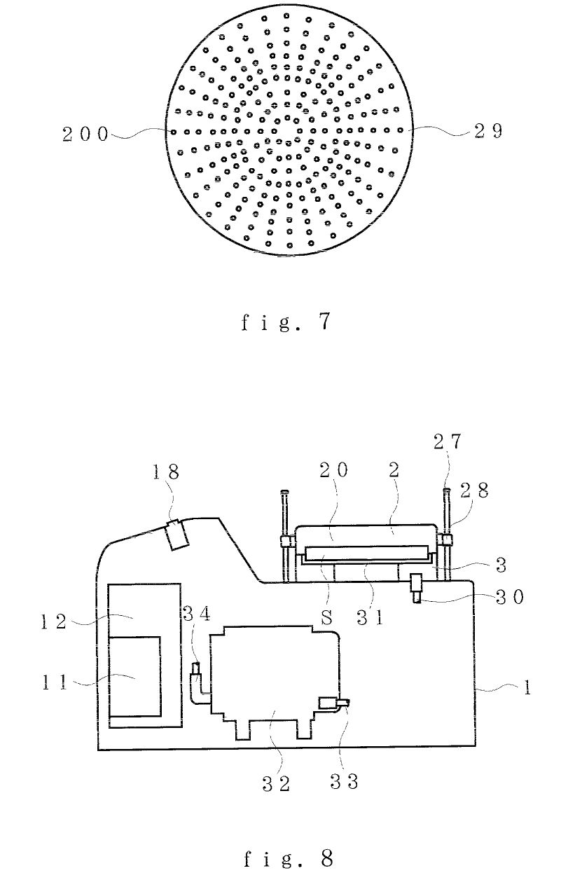

FIG. 8 shows the capturing apparatus of a third embodiment. Linear guides 27 comprised of steel balls are provided upright on the upper surface of capturing apparatus 1 at symmetrical locations about both ends of cylindrical chamber 3. Sliding cylinders 28 provided on both sides of cap 2 fit in chamber 3 pass through the above-mentioned linear guides 27. The diameter of sliding cylinders 28 is such that they are slightly tight with respect to the linear guides 27. Namely, the diameter of sliding cylinders 28 is set such that, although cap 2 can be raised up by pulling upward, cap 2 does not drop down due to its own weight. Furthermore, sets of these linear guides 27 and sliding cylinders 28 may also be provided at three or four symmetrical locations. Furthermore, reference symbol 18 is a timer switch for switching vacuum pump 32 on and off.

Thus, in the capturing apparatus of this embodiment as well, problems do not occur resulting from porous plate 20 falling out of cap 2 and dropping onto the floor thereby resulting in contamination of the floor by miscellaneous microorganisms and so forth. Furthermore, a storage compartment able to house multiple types of porous plates may also be provided within the capturing apparatus.

Although the above has provided an explanation of three types of embodiments of the present invention, according to this invention, since the problem of contamination of a desk and so forth caused by a porous body coming in contact with that desk and so forth when transferring a Petri dish or other container has been eliminated, the usefulness of this invention has been proven.

What is claimed is:

1. An apparatus for capturing suspended microorganisms comprising: a cap provided so as to be able to be opened and closed freely in a chamber without being separated from said chamber that serves as both an inlet port for outside air and a transfer port for a Petri dish or other container; a porous plate provided in said inlet port of said cap; a holder for said container provided at a position that allows receiving of outside air aspirated from said porous plate; and, a deaerating pump for generating negative pressure in said chamber.

2. An apparatus for capturing suspended microorganisms according to claim 1 wherein a large number of pinholes in said porous plate are arranged in the form of a grid.

3. An apparatus for capturing suspended microorganisms comprising: a cap that is maintained roughly in the horizontal position at all times in a chamber, is provided so as to be opened and closed freely without being separated from said chamber and serves as both an inlet port for outside air and a transfer port for a Petri dish or other container; a porous body provided in said inlet port of said cap; a holder for said container provided at a position that is able to receive outside air aspirated from said porous body; and, a deaerating pump for generating negative pressure in said chamber.

4. An apparatus for capturing suspended microorganisms according to claim 3 wherein said cap is attached to a chamber by a parallel linkage.

5. An apparatus for capturing suspended microorganisms according to claim 3 wherein a large number of pinholes in said porous plate are arranged in the form of a grid.
